(19) United States
(12) Patent Application Publication
(10) Pub. No.: US 2026/0212496 A1
(43) Pub. Date: Jul. 23, 2026

(54) METHOD FOR ASSISTING WITH THE CLASSIFICATION OF A HISTOLOGICAL SECTION FROM A PATIENT, ASSOCIATED DEVICE

(71) Applicants: TRIBUN HEALTH, Paris (FR); ASSISTANCE HOPITAUX DE PARIS (AP-HP), Paris (FR)

(72) Inventors: Cécile BADOUAL, Paris (FR); Yaëlle BELLAHSEN, Paris (FR); Mélanie LUBRANO, Paris (FR); Thomas WALTER, Paris (FR)

(73) Assignees: TRIBUN HEALTH, Paris (FR); ASSISTANCE HOPITAUX DE PARIS (AP-HP), Paris (FR)

(21) Appl. No.: 19/139,448

(22) PCT Filed: Dec. 19, 2023
(86) PCT No.: PCT/FR2023/052048

§ 371 Date: Jun. 16, 2025

(30) Foreign Application Priority Data
Dec. 19, 2022 (FR) FR2213863

(51) Int. Cl. G06T 7/00; G06V 10/82; G06V 20/69; G16H 10/40; G16H 30/40

(52) U.S. Cl. CPC G06T 7/0012; G06V 10/82; G06V 20/698; G16H 10/40; G16H 30/40; G06T 2207/30024

(57) ABSTRACT

A computer-implemented method for assisting with the classification of a histological section from a patient into one of N classes with a predefined order, a computer device and an associated computer program product.

### FIELD

The present invention relates to the field of digital pathology, in particular the classification of histological sections.

The present invention relates more particularly to a computer-implemented method for assisting with the classification of a histological section from a patient and an associated device.

### BACKGROUND

Histological classification methods (or grading) are used to define the appearance of histological lesions compared to that of healthy tissue. In the case of precancerous lesions, grading helps to predict the potential of a lesion to evolve into an invasive cancer, to better guide medical management. In particular, it is estimated that the detection and evaluation of dysplastic lesions of the head and neck could prevent almost 90% of invasive cancers. Pathologists also use grading to estimate the patient's prognosis and guide treatment choices. Examples of cancers are head and neck squamous cell carcinomas (HNSCC) or cervical cancer. Such pathologies are a major public health problem due to their high mortality rate and the morbidity related to their treatment or late diagnosis.

Despite these challenges, classifying lesions according to their severity remains a complex exercise. For example, the classification of head and neck dysplasia has been a very controversial topic for many years. Since the first classification proposed by Kleinsasser in 1963, many different classifications have been proposed by expert pathologists without reaching a strong consensus. Each used different terminology and classification methods, but their reproducibility has always been low to moderate.

In order to improve the reliability of classification, the World Health Organisation (WHO) recommends classifying squamous laryngeal dysplasia into only two categories: mild and severe. The severe category includes moderate, severe dysplasia and carcinomas in situ.

For the oral cavity, the WHO classification system has retained the distinction between moderate and severe lesions. Interestingly, it is the only classification system that retains three distinct gradings, even though oral squamous lesions share the same pathophysiology and are induced by the same carcinogens as in the larynx, hypopharynx, trachea, and lateropharyngeal space.

Nevertheless, the reproducibility of classifications between pathologists remains limited for all grading methods. This classification difficulty can be explained by the multiplicity of elements to be taken into account, both at the cytological and architectural level, on an epithelium that may show significant variations in thickness depending on the anatomical location, and the presence of inflammatory and dystrophic changes that can sometimes be difficult to distinguish from real dysplasia.

Furthermore, the application of arbitrary classification categories to a continuous spectrum of lesions without absolute or clearly definable limits leads to more subjectivity in the assignment of a category by a pathologist.

Thus, new tools are needed to help pathologists achieve robust and consistent classifications in different areas that require lesion grading, such as squamous lesions of the head and neck and cervix, dysplasia in the digestive system, or metabolic steatohepatitis (or NASH), for better therapeutic management.

Recently developed techniques include digital pathology, which allows data to be managed based on information generated from digitised sample slides.

In particular, artificial intelligence has been successfully applied to digital pathology, for example to detect metastases, to determine the subtype of cancer or to estimate the prognosis of patients. Several studies have even shown the positive benefits of AI tools when combined with the expertise of pathologists. However, these algorithms are not yet widely accepted in clinical practice as standalone diagnostic tools due to concerns about their instability on external cohorts, their sensitivity to domain changes, and their lack of interpretability.

Currently, machine learning models also give predictions independently of histological ambiguity, without providing information on the confidence of the predictions, even if the sample is difficult to analyse for pathologists themselves. In such ambiguous cases, a pathologist may ask for a second opinion or additional analysis, whereas artificial intelligence would not report any uncertainty.

The confidence (or the equivalent of “uncertainty”) of machine learning models has been studied in recent years and applied to the medical context. However, very little work has been done on histopathology applications. Early work focused on model performance for segmentation tasks, active learning, or out-of-distribution (OOD) sample detection.

Methods for measuring the confidence of machine learning models include the use of the softmax output of the model, the Monte Carlo dropout method, the use of deep network ensembles, or test time augmentation. As a reminder, deep learning dropout is a technique consisting of randomly deactivating certain neurons in a neural network layer during training in order to regularise the network.

None of these methods have been developed to take into account the ordered aspect of classes, which is essential and rich information for the development of a grading model.

Furthermore, artificial intelligence models for WSI (whole slide image) classification are difficult to train due to the large size of histological data sets. The most common methods evaluated in these studies are computationally resource-intensive because they rely on the generation of a distribution of predictions, and are therefore not suitable for data sets that contain thousands of histological sections and millions of tiles (or sub-images).

The invention falls within this context.

### SUMMARY

A first aspect of the invention relates to a computer-implemented method for assisting with the classification of a histological section of a patient into one of N classes ordered according to a predefined order, each class corresponding to a degree of severity of a pathology, N being an integer strictly greater than 2, by means of a confidence index representative of a degree of classification reliability, the method comprising:
a step of implementing a machine learning model in order to classify said image in one of N ordered classes, said model returning as output N classification probabilities each associated with one of the N ordered classes,
a step of calculating said confidence index, based on a distance between the N classification probabilities.

Thus, the calculated confidence index allows a user, such as a pathologist, to have information on the reliability of the classification obtained by implementing the machine learning model. Furthermore, the definition of the confidence index is such that it reflects the degree of confidence of a pathologist when performing a manual classification task.

In other words, the invention provides a measure of uncertainty designed for classification tasks, which does not require additional training or additional inferences. This measure of uncertainty is easy to interpret because it quantifies the hesitation of a model between two classes, and is therefore particularly suitable for classification tasks.

This grade-sensitive (i.e. classification-sensitive) confidence index allows histological sections of high-confidence to be accurately separated from those of low-confidence and thus establishes a correspondence with the behaviour of pathologist, as will be seen in the following. In this way, such a confidence index can provide useful information to pathologists, thus helping them to better understand how the machine learning model works, and to standardise their diagnoses.

In some embodiments, the machine learning model has been previously trained using a cost function weighted by a plurality of coefficients representative of a risk of incorrect classification. Thus, advantageously, the higher the risk of incorrect classification, the greater the cost function, reflecting an unreliable classification. Conversely, the lower the risk of incorrect classification, the lower the cost function, reflecting a reliable and optimal classification.

In one embodiment, one coefficient of the plurality of coefficients has a higher value in a first case of incorrect classification into a class that is farther from the correct class, according to said predefined order, than in a second case of incorrect classification into a class less far from said correct class, according to said predefined order, than in the first case. Advantageously, the cost function defined in the present invention makes it possible to obtain an ordered output from the machine learning model. In other words, the output values of the machine learning model (i.e. the probabilities) are ordered from the most likely to the least likely class according to the predefined order (for example, here: the severity of the lesions). For example, if the highest probability is obtained for class 3, the second will be class 2, then 1 and then 0. If the highest probability is class 2, the second will be class 3 or class 1, then class 1 or class 3, then class 0.

In particular, the present invention advantageously makes it possible to help classify a histological section, by providing two pieces of information: a prediction of class, selected from N classes ordered according to a predefined order, and a confidence index of said prediction. This is made possible by the specific cost function, which enables the learning model to be trained to minimise an ordered risk prediction, which is an essential requirement for pathologists, as it penalises more errors with high medical impact (i.e. a prediction of a class that is not adjacent to the actual class associated with the training data) and by the confidence index which also reflects a greater hesitation of the model if the difference in probabilities (or risks) between the 2 most probable classes is small (these two classes being, due to the cost function, always adjacent).

In some embodiments, the machine learning model is implemented using said cost function and such that each of the plurality of assignment probabilities is associated with a risk of incorrect classification, said risk varying increasingly with said cost function.

In one embodiment, the cost function is defined by the formula:

$$L = {\sum }_{i} {\sum }_{j} ln ( 1 + exp ( {2}_{i , j} ( {\hat{c}}_{i} - {p}_{i , j} ) ) )$$

where ĉᵢ represents the predicted cost for class Cᵢ, and p_{i,j} represents a coefficient of a cost matrix, and more specifically: p_{i,j} is the coefficient if the true class is Cᵢ and the prediction is Cⱼ, and 2_{i,j}=−1_{i≠j}+1ᵢ₌ⱼ.

In some embodiments, the distance is a difference between the two highest of the N classification probabilities.

Thus, the confidence index will be representative of the distance between the two highest classification probabilities. The further these two probabilities are apart, the higher the confidence index and the higher the degree of reliability of the classification. The closer these two probabilities, the lower the confidence index will be, reflecting uncertainty as to the classification in either of these two highest probabilities.

In some embodiments, the step of implementing the machine learning model comprises:
a step of dividing said image into a set of M sub-images (i.e. tiles),
for each sub-image, an extraction of a plurality of characteristics,
for each sub-image, a score is calculated, based on the plurality of extracted characteristics,
classifying said image into one of the N ordered classes, based on the plurality of characteristics and the scores of the sub-images.

In some embodiments, the machine learning model is a multiple instance learning model. Indeed, whole slide images (WSI), are very large images. Thus, a phase of dividing into sub-images makes it possible to process the latter by reducing the size of the elements to be stored in a computer memory.

In some embodiments, the histological section comprises dysplastic lesions.

In some embodiments, the dysplastic lesions are squamous lesions of the upper aero-digestive tracts.

In other embodiments, dysplastic lesions are gastric lesions.

In other embodiments, dysplastic lesions are cervical lesions.

In other embodiments, the N classes ordered in a predefined order are each representative of a Gleason score for the evaluation of prostate cancer.

In other embodiments, the N classes ordered in a predefined order are representative of biomarker scores or histological lesions.

In some embodiments, calculating the score of each sub-image comprises implementing an attention mechanism. This implementation is advantageous because it does not require a manual annotation step. In this way, the machine learning model independently learns to assign an importance score to each sub-image. In other words, the model is advantageously an unsupervised or minimally supervised model.

In some embodiments, the method comprises, after the step of calculating said confidence index, a step of comparing said confidence index with a threshold value determined on the basis of a metric representing a reliability of the machine learning model calculated after the implementation of said model on a database of validation images, said metric preferably being an area under the curve (AUC) value.

In some embodiments, the method further comprises, after the comparison step, a step of automatically assigning a degree of analysis priority by an experienced user, the degree of analysis priority being:
a degree of high priority if the confidence index is below the threshold value,
a degree of low priority if the confidence index is above the threshold value.

In some embodiments, the method further comprises a step of automatically assigning a degree of analysis priority by a user, the degree of analysis priority being:
a degree of high priority if said histological section is classified into a class corresponding to a high degree of severity,
a degree of low priority if said histological section is classified into a class corresponding to a low degree of severity.

In some embodiments, the histological section has been manually classified into one of the N ordered classes, referred to as the manual class, by a user, prior to the step of implementing the machine learning model. In this case, the method further comprises a step of comparing the manual class with the class obtained during the step of implementing the machine learning model.

A second aspect of the invention relates to a computer device comprising a circuit configured to implement a method as described above.

A third aspect of the invention relates to a computer program product including instructions for implementing steps of the method as described above, when this program is executed by a processor.

The present disclosure also relates to a non-transitory computer-readable recording medium comprising instructions which, when executed by a computer, cause it to implement a method for assisting with the classification of a histological section, according to any one of the embodiments described above.

Such a non-transitory computer-readable recording medium may be, without limitation, an electronic, magnetic, optical, electromagnetic, infrared or semiconductor device, or any suitable combination of the above. It should be noted that the following examples, although more specific, are only an illustrative and non-exhaustive list, easily assessed by a person with ordinary knowledge of the art: a laptop floppy disk, a hard disk, a ROM, an EPROM (Erasable Programmable ROM) or a flash memory, a portable CD-ROM (Compact-Disc ROM).

### BRIEF DESCRIPTION OF THE FIGURES

FIG. 1 is an example of a flowchart illustrating a computer-implemented method for assisting with the classification of a histological section of a patient according to one or more embodiments.

FIG. 2 is an example of a flowchart illustrating the sub-steps of a method step illustrated in FIG. 1.

FIG. 3 is a representation of a digitised histological section, on the left, and the division of this digitised histological section into sub-images.

FIG. 4 is an example of a flowchart illustrating the sub-steps of a step of the method illustrated in FIG. 1.

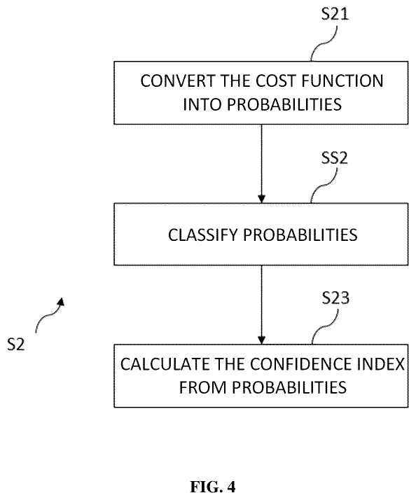

FIG. 5 illustrates a device for implementing a method such as those illustrated in FIG. 1, FIG. 2 and FIG. 4.

FIG. 6 shows the change of the AUC metric (area under the ROC curve) over all 128 histological sections of the validation image database according to the threshold value to which the confidence index I is compared.

FIG. 7 shows the overall change of the AUC over the standard reference test set as the most uncertain sections are removed. The overall AUC was measured (mean over the 4 classes) on the same number of sections, after removing the most uncertain sections from the test set. The faster the AUC increases, the better the sections selected.

FIG. 8 shows the distribution of confidence scores in relation to pathologists' agreements

### DETAILED DESCRIPTION

A first aspect of the invention relates to a computer-implemented method 100 for assisting in the classification of a histological section from a patient into one of N classes ordered according to a predefined order, by means of a confidence index representative of a degree of classification reliability. N is an integer strictly greater than 2. Each of the N classes corresponds to a degree of severity of a condition. For example, the pathology may be head and neck dysplasia, cervical lesions, gastric lesions, or cervical lesions.

Typically, the histology section is a digital image of a sample slide of the patient. For example, the sample comes from a biopsy. In another example, the sample is a surgical sample.

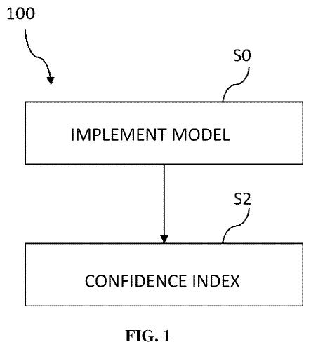

FIG. 1 is a flowchart representing different steps according to one embodiment of the method 100 to assist with the classification of a histological section into a class Cᵢ, i being an integer between 0 and N−1. Class C₀ may represent the class associated with the absence of lesions.

According to this embodiment, in step S0, a machine learning model is implemented in order to classify the histological section in a class Cⱼ among the N classes Cᵢ. The model returns, at the end of step S0, N classification probabilities, each associated with one of the N classes Cᵢ. Class Cⱼ is associated with the highest classification probability.

In a step S2, a confidence index I is calculated based on a distance d between the N classification probabilities. For example, the distance d is the difference between the two highest probabilities Pₖ, Pₗ among the N classification probabilities.

A user, such as a pathologist, can then take into account the value of the confidence index to assess the reliability of the classification in class Cⱼ obtained at the end of step S0.

Step S0 of implementing the machine learning model will be described in more detail below.

In some embodiments, the machine learning model is a multi-instance learning model (MIL). Multi-instance learning aims to assign labels to sets or bags of instances, as opposed to traditional supervised learning where each instance is assumed to be independent and distributed equally and has to be labelled individually. The MIL model used in these embodiments comprises a characteristic extractor module, a scoring module, and a classification module.

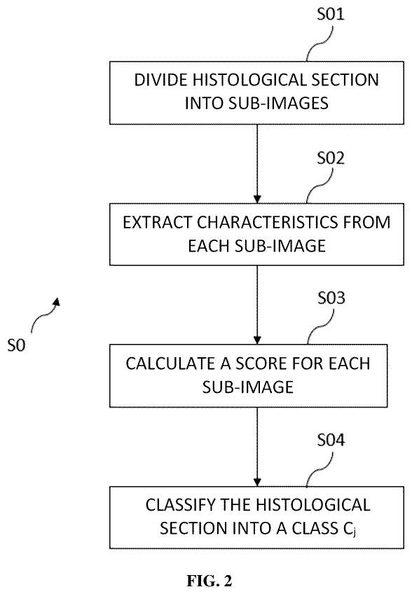

FIG. 2 is an example of a flowchart representing the sub-steps performed during step S0.

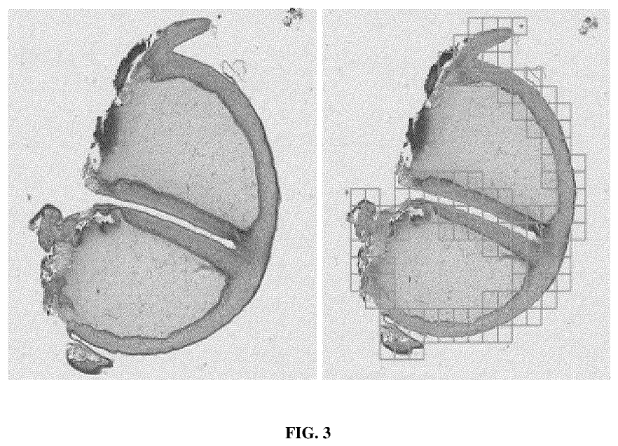

In sub-step S01, the histological section Iₘ to be classified may be divided into a set of M sub-images. The sub-images are the instances of the MIL model and together form a bag of instances. FIG. 3 shows, on the left, a histological section, and on the right, a set of sub-images dividing this histological section.

In sub-step S02, for each of the M sub-images, a plurality of characteristics are extracted by the characteristic extractor module. For example, the characteristic extractor module is a Dense Convolutional Network. In a convolutional neural network, each layer receives information from all preceding layers, and each layer transmits its characteristics to all subsequent layers. For example, for each of the M sub-images, a characteristic vector of size 1024 is obtained.

In a sub-step S03, for each of the M sub-images, the scoring module calculates a score based on the plurality of characteristics extracted. The score is representative of the importance of a sub-image in the classification of the histology section. For example, the scoring module calculates scores using an attention mechanism. The attention mechanism consists of one or more layers of dense neurons, inputting the extracted characteristic vector and outputting a normalised score of between 0 and 1 attesting to the importance of the input object. This importance score is therefore learned autonomously by the network during the model training process.

In sub-step S04, the classification module classifies, based on the plurality of characteristics and the scores of the M sub-images, the histological section Iₘ into a class Cⱼ among the N classes Cᵢ. For example, the classification module aggregates the scores obtained in sub-step S03 and the characteristic vectors of each sub-image obtained in sub-step S02 in order to obtain a representation of the histological section. The representation is, for example, a weighted sum of characteristic vectors with the score of the corresponding sub-image as the weight.

It is described in the following how, when the machine learning model used in step S0 is a multiple instance learning model (MIL), it is trained during a training phase prior to its use in the method 100.

### Training Phase:

An example of a training phase is described here, where the pathology of the histological section to be classified consists of dysplasias of the head and neck. In this example, the number N of classes selected is equal to 4, with the following 4 classes:
class 0: benign,
class 1: presence of low-grade dysplastic lesions,
class 2: presence of high-grade dysplastic lesions,
class 3: presence of invasive carcinoma.

The characteristic extractor module is initialised with weights obtained by pre-training by self-supervised learning. For example, the SimCLR architecture can be used. SimCLR architecture involves training a convolutional network using unsupervised images. The training process is performed by generating for each image 2 slightly modified images in a random manner (rotation, colour alteration, zoom) then forcing the SimCLR network to move similar images closer together and move different images further apart within the group of modified images (which is referred to as the contrastive task).

A training database of 2144 digitised histological sections is used, the classification of which in one of the four preceding classes is known. The training database includes a small number of class 1 histological sections (229 histological sections, i.e. 10.7%). Classes 0, 2 and 3 are equally represented.

Each histology section in the training database is divided into sub-images. For example, each sub-image has a size of 224×224 pixels with a resolution of 1 micron per pixel. For example, after splitting into sub-images, the background of each histology section is removed. Thus, for the database as a whole, an average of 1300 sub-images per histological section is obtained.

In order to reduce processing time, sub-images that do not contain epithelial cells are removed using a binary segmentation algorithm, separating the epithelium and any carcinoma from other tissues. The binary segmentation algorithm may for example be an U-Net-type artificial neural network (otherwise referred to as a “fully convolutional network”) trained on 5439 annotated 512×512 pixel sub-images from 121 histological sections. “Annotated” means, for example, that each pixel of the sub-image is associated with a label. The label can be determined or validated by experts such as pathologists or biologists, and it represents the “truth”.

It should be recalled that training a learning model consists of determining a set of model parameters and/or teaching the model a prediction function from the training data, so that the model can then predict the label of a new piece of received data (in the present application, predict the class of a new received histological section).

During the training (or “learning”) phase of the learning model, the model parameters are estimated iteratively in order to minimise an objective function related to a cost function L, which represents the prediction error rate that the model achieves on the training data.

According to the present description, the cost function used herein is weighted by a plurality of coefficients representative of a risk of incorrect classification. The plurality of coefficients constitutes a cost matrix.

More specifically, the advantage is the ordered nature of the N classes. One of the plurality of coefficients of the cost matrix has a higher value in a first case of incorrect classification in a class further from a correct class, according to the predefined order, than in a second case of incorrect classification in a class less far away from said correct class, according to the predefined order, than in the first case. In other words, the greater the difference (i.e. the error) between the predicted class and the actual class (the “true” class), the greater the value of the cost function. An error in classifying an image belonging to class C₂ into class C₁ will correspond to a lower value of the cost function than an error in classifying an image belonging to class C₃ into class C₀.

For example, the cost function L can be written as:

$$L = {\sum }_{i} {\sum }_{j} ln ( 1 + exp ( {2}_{i , j} ( {\hat{c}}_{i} - {p}_{i , j} ) ) )$$

where ĉᵢ represents the predicted cost for class Cᵢ, and p_{i,j} represents a coefficient of the cost matrix mentioned above, and more specifically: p_{i,j} is the coefficient if the true class is Cᵢ and the prediction is Cⱼ, and 2_{i,j}=−1_{i≠j}+1ᵢ₌ⱼ.

Table 1 shows an example of a plurality of coefficients fulfilling the preceding condition:

TABLE 1
coefficients of a cost function representative
of a risk of incorrect classification

| | Class 0 | Class 1 | Class 2 | Class 3 |
|---|---|---|---|---|
| Truth | (prediction) | (prediction) | (prediction) | (prediction) |
| Class 0 | 0.0 | 0.1 | 0.7 | 1.0 |
| Class 1 | 0.1 | 0.0 | 0.3 | 0.7 |
| Class 2 | 0.7 | 0.3 | 0.0 | 0.3 |
| Class 3 | 1.0 | 0.7 | 0.3 | 0.0 |

For example, according to the cost matrix in Table 1, if the truth is class C₀, and the predicted class is class 2, the cost coefficient is C_{0,2}, equal to 0.7.

Thus, by using such a cost function, the MIL model is trained to predict a risk of class-dependent incorrect classification. The class obtained by the classification by the model corresponds to the class minimising the risk of incorrect classification.

Step S2 of calculating the confidence index I will now be described in more detail. The confidence index I is representative of a degree of reliability of the classification obtained in step S0. In other words, the confidence index represents an indicator for pathologists of the confidence they may have in the result of a predicted classification obtained in step S0. The confidence index I is obtained by a succession of sub-steps. FIG. 3 is an example of a flowchart representing the sub-steps performed during step S2.

In a first sub-step S21, the values of the cost function L corresponding to the N classes are converted into probabilities by applying a decreasing function to the cost function. For example, the softmax function is applied to the opposite of the cost function values in order to obtain probabilities. As seen above, for the specific choice of the cost function described above, a low cost value corresponds to a low risk of incorrect classification, and thus a high probability of correct classification. A high value for the cost function corresponds to a high risk of incorrect classification, and thus a low probability of correct classification.

In a second sub-step S22, the different probabilities are ranked, in ascending or descending order.

In a third sub-step S23, the confidence index is calculated by the difference between the values of the two highest probabilities.

Thus, the further apart the two highest probabilities, the higher the confidence index I and the more reliable the classification by the model. If the two highest probabilities are close, this reflects a hesitation between two close classes, and the confidence index is then representative of this uncertainty. The confidence index thus makes it possible to highlight ambiguous or difficult cases.

In some embodiments, the confidence index I is compared to a threshold value. For example, the threshold value is determined on the basis of a metric representing a reliability of the machine learning model calculated after implementation of the model on a validation image database, the metric preferably being an area under the ROC curve value. When the confidence index I is higher than the threshold value, the degree of reliability of the classification obtained with the model is high, whereas when the confidence index is lower than the threshold value, the classification obtained with the model is uncertain.

Advantageously, the confidence index I can be used to automate the workflow of pathologists.

Thus, in some embodiments, the confidence index I may be used to define priorities in histological section analysis tasks by a pathologist.

For example, when the confidence index I is compared to a threshold value, a degree of prioritisation can be automatically assigned to each histological section classified with the model. A high degree of prioritisation can be assigned to histological sections of uncertain classification, for which expert opinion is required, whereas a low degree of prioritisation can be assigned to histological sections with a high degree of classification reliability and thus assigned to less experienced pathologists.

In another example, a degree of prioritisation can be automatically assigned according to the degree of severity corresponding to the class determined by the model. Thus, a histological section determined by the model as having a high degree of pathology severity will be assigned a high degree of prioritisation and the pathologist will treat this histological section as a priority. A histological section determined by the model as having a benign degree of severity and a high confidence index I will be assigned a low degree of prioritisation, and the histological section will be processed after the priority histological sections. Alternatively, it may be decided not to assign such a histological section, due to the high certainty in the classification determined by the model, thus reducing the workload of the pathologists.

In another example, three levels of prioritisation can be defined and assigned as follows. Thus, a high degree of prioritisation can be assigned to a histological section classified according to a high degree of severity and having a high confidence index I. An average degree of prioritisation may be assigned to a histological section with a low confidence index I, regardless of the degree of severity of the class determined by the model. Finally, a priority level of zero (i.e. for which the histological section will not be manually reviewed by a pathologist) may be assigned to a histological section classified as having a low degree of severity and a high confidence index I.

Advantageously, the confidence index I can be used to assist pathologists in their diagnosis. Thus, a pathologist may choose whether or not to take into account the classification determined by the model according to the value of the confidence index I.

Advantageously, the confidence index I can be used to distribute the workload of the pathologists of the same unit. Thus, histological sections with a high confidence index I will be considered easy to analyse, and may be assigned to pathologists with a junior profile, whereas histological sections with a low confidence index I will be considered more difficult to analyse, and may be assigned to more experienced pathologists.

Advantageously, the confidence index I can be used for automated revision tasks. Sometimes a pathologist may ask for a second opinion on a histological section that is particularly difficult to analyse. This revision work may be performed by implementing the automatic classification method with the model according to the invention.

Advantageously, the confidence index I can be used to streamline the work of pathologists. Thus, histological sections with a high confidence index I can lead to rapid medical decisions. Conversely, histological sections with a low confidence index I may give rise to additional tests.

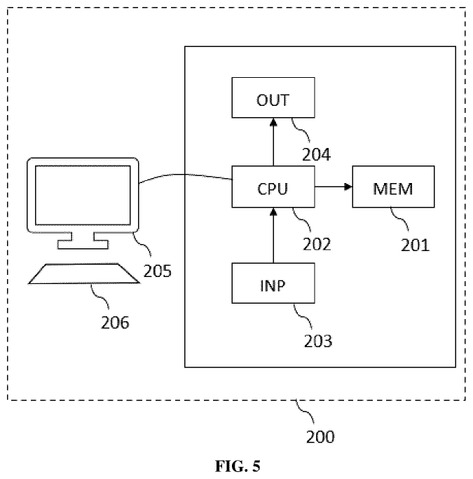

FIG. 5 illustrates a device 200 for implementing the method 100 for assisting in classifying a histological section of a patient into one of N classes ordered according to a predefined order according to one or more embodiments.

The device 200 may comprise a memory 201 for storing instructions for implementing steps of the method 100 for assisting in classifying a histological section of a patient, the data received, in particular the histological section to be classified, the data from the training database, and temporary data for performing all or some of the steps of the methods described above.

The device 200 further comprises a control circuit 502, an input interface 203 for receiving data, including the histological section to be classified, the training database data, and an output interface 204 for providing output data, such as parameters of the learning model or a confidence index I.

In one or more embodiments, to enable easy interaction with a user, the device 200 may be in the form of a computer including a screen 205 and a keyboard 206. The device 200 may be a mobile terminal, a computer, a network of computers, an electronic component, or another apparatus comprising a processor operatively coupled to a memory, as well as, according to the chosen embodiment, a data storage unit, and other associated hardware such as a network interface and a media drive to read and write to a removable storage medium. The removable storage medium can be, for example, a compact disk (CD), a video/digital versatile disk (DVD), a flash disk, a USB stick, etc. Depending on the embodiment, the memory, data storage unit, or removable storage medium contains instructions that, when executed by the control circuit 202, cause this control circuit 202 to control the input interface 203, output interface 204, and memory 201.

Furthermore, the block diagram shown in FIG. 1 is a typical example of a program in which certain instructions can be performed by the device described. As such, FIG. 1 may correspond to the flowchart of the general algorithm of a computer program in a particular embodiment.

### EXAMPLES

The present invention will be better understood on reading the following examples which illustrate the invention without limitation.

### Example 1

This example illustrates the confidence index results obtained on a validation image database (“gold standard test set”) and shows how the confidence index I calculated in step S2 of the method 100 is representative of the similarity between automatic classification with the model in step S0 and a manual classification performed by pathologists. The validation image database includes biopsy samples. The images in the database have been chosen so that all classes are fairly represented.

The validation image database is derived from a selection of 128 histological sections from a more general database denoted BD, the selection having been made so that each image in the validation image database can be considered independent of the others.

The database BD includes digitised histological sections of patients treated between 2000 and 2013 at the Georges Pompidou European Hospital in Paris, France. Corresponding patients were diagnosed with dysplasia or carcinoma of the head and neck, larynx, pharynx, nasopharynx, hypopharynx, lateral edge of the tongue, or oral cavity. The resolution of the digitised histological sections was 0.45 microns per pixel. Each digitised histology section was manually classified according to the WHO classification.

More specifically, an independent classification by two pathologists based on validation image data was performed, manually. The result obtained by the two pathologists was consistent on 79 digitised histological sections. A consensus was reached, during a review, on 49 of the remaining 52 histological sections. Thus, a total of 128 histology sections were selected for the validation image database.

### Results

The MIL model according to several embodiments was used to classify the digitised histological sections in the validation image database. The area under the ROC curve metric, representative of the reliability of a classifier, was calculated. The mean value of the area under the ROC curve for the four classes C₀, C₁, C₂ and C₃ obtained is 0.878. Seven digitised histological sections belonging to class C₁ were classified in class C₀. Four histological sections belonging to class C₃ were incorrectly classified, predicting class C₁ or C₂.

By setting a threshold value of 0.5 for the confidence index I, 52 digitised histological sections (40.6% of the validation image database) were considered uncertain, most of them belonging to class C₁. For the remaining digitised histology sections, the area under the ROC curve was 0.987. No histological sections belonging to class C₃ (corresponding to the presence of carcinoma and therefore the highest degree of severity) were incorrectly classified.

### Example 2

This example illustrates the performance of the MIL model used in step S0 of the method 100 applied to the 128 histological sections of the validation image database.

Table 2 summarises the values of the area under the ROC curve metric of the model.

TABLE 2
Model performance

| | Mean AUC | AUC Class 3 | AUC Class 0 |
|---|---|---|---|
| Model vs “truth” | 0.886 | 0.965 | 0.954 |
| | [0.822-0.939] | [0.920-1.000] | [0.907-0.989] |
| Model vs “truth” | 0.931 | 0.987 | 1.000 |
| on “certain” | [0.892-0.965] | [0.962] | [1.000-1.000] |
| histological | | | |
| sections | | | |
| (threshold of 0.5) | | | |
| Model vs “truth” | 0.764 | 0.904 | 0.911 |
| on “uncertain” | [0.672-0.848] | [0.771-0.997] | [0.822-0.979] |
| histological | | | |
| sections | | | |
| (threshold of 0.5) | | | |

### Example 1

This example illustrates the performance of the machine learning model applied to the 128 histological sections in the validation image database, independent of the confidence index I. Table 3 illustrates this performance with the area under the ROC curve values of the model.

TABLE 3
Model performance with AUC metric (area under ROC curve)

| Mean | AUC [95% confidence index] |
|---|---|
| | 0.886 [0.822-0.939] |
| Class 0 (benign) | 0.909 [0.848-0.958] |
| Class 1 (low grade) | 0.807 [0.718-0.883] |
| Class 2 (high grade) | 0.859 [0.786-0.922] |
| Class 3 (invasive carcinoma) | 0.97 [0.936-0.995] |

Another way of representing this performance is to count the histological sections that have been well classified by model prediction. This other representation is carried out for example by means of a so-called confusion matrix. The closer the confusion matrix is to a diagonal matrix, the better the corresponding model is.

Table 4 shows the confusion matrix obtained for the 128 histological sections of the validation image database.

TABLE 4
Confusion matrix

| | | | | | |
|---|---|---|---|---|---|
| Truth | Class 0 | 19 | 7 | 3 | 0 |
| | Class 1 | 7 | 10 | 6 | 0 |
| | Class 2 | 1 | 10 | 20 | 3 |
| | Class 3 | 0 | 2 | 2 | 38 |
| | | Class 0 | Class 1 | Class 2 | Class 3 |

Prediction by model

### Example 4

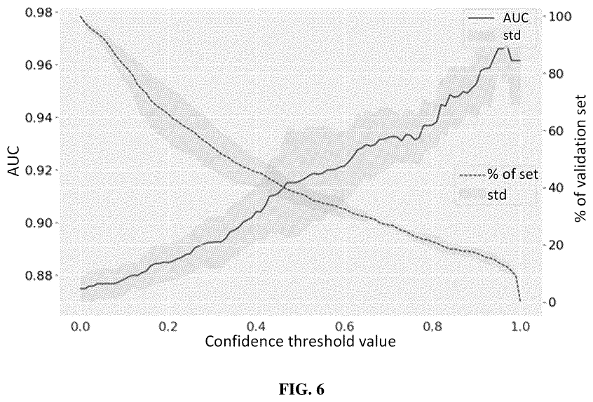

This example illustrates the effect, when the confidence index I is compared to a threshold value, of the choice of this threshold value on the classification performance. FIG. 6 shows the change of the AUC metric (area under the ROC curve) for all 128 histological sections in the validation image database as a function of the threshold value against which the confidence index I is compared. It can be seen that as the threshold value increases, the number of histological sections considered uncertain increases and are removed from the data set, as can be seen on the dotted curve. It is also observed that the AUC increases when the most uncertain histological sections are removed.

### Example 5

This example illustrates the similarity between the value of the confidence index I calculated and the degree of confidence pathologists have in their classification when performing a manual classification task.

Table 5 gives the number and percentage of histological sections estimated as certain and uncertain by the confidence index compared to the “manual” confidence level. Manual confidence level refers to the agreement between the class of the histological section assigned during a first manual classification task performed by a first pathologist and the class reassigned after review by a panel of pathologists.

TABLE 5
Similarity of the confidence index with the
degree of confidence “manual”

| | Number of high | Number of low |
|---|---|---|
| | confidence histology | confidence histology |
| Threshold: 0.5 | sections “manual” | sections “manual” |
| I > 0.5 | 60 (79%) | 16 (21%) |
| (high reliability) | | |
| I < 0.5 | 30 (58%) | 22 (42%) |
| (low reliability) | | |

Of course, the present invention is not limited to the embodiments described above.

### Example 6

### Minimally Supervised WSI Classification of Precancerous Lesions of the Head and Neck

Classifying precancerous head and neck lesions is a difficult task: often, the boundaries between each grade are blurred and the related histological models overlap. Moreover, several grades of lesions can coexist on the same WSI. This example focuses on classifying precancerous and cancerous lesions of the head and neck according to their severity. These lesions are classified by evaluating several morphological and cytological abnormalities of the tissue according to WHO recommendations. The head and neck epithelium may be either normal/benign (class 0), or have a low-grade lesion (class 1), a high-grade lesion (class 2), or an infiltrating (i.e. invasive) carcinoma (class 3).

To assess the benefit of the confidence measurement in the context of a classification task, an internal data set consisting of precancerous and cancerous tissue samples of the head and neck was used. The data set consisted of 2121 hematoxylin and eosin (H&E) sections. Each section was labelled with the most severe lesion it contained (grade 0 to 3). Given the inherent ambiguity of these histological patterns, a standard test set of 128 sections was blindly reviewed by 2 pathologists, and a consensus diagnosis was established.

An architecture derived from the MIL attention model was used. Each WSI was cut into small tiles and supplied to the input of a neural network, according to the invention. A global label associated with the WSI was used to train the model. An attention mechanism was used to detect the tiles that are most relevant for the classification.

To take advantage of the ordinal nature of the classes (i.e. classes ordered according to a predefined order), the cost aware classification loss function was used: the smooth one-sided regression (SOSR) loss. The output from the network is a specific risk associated with the class Ĉ rather than an a posteriori probability. No softmax function was used. The predicted class corresponded to the class minimising this risk. The cost function was defined as follows:

$$L = {\sum }_{i} {\sum }_{j} ln ( 1 + exp ( {2}_{i , j} ( {\hat{c}}_{i} - {C}_{i , j} ) ) )$$

where 2_{i,j}=−1_{i≠j}+1ᵢ₌ⱼ, ĉᵢ is the i-th coordinate of the network output and C is the error table. Using ordinary risk predictions is a crucial requirement for pathologists, as it penalises errors with a high medical impact. However, if a normal classification setting with cross-entropy is used, serious classification errors are more likely to occur. In this example, the error table is defined as proposed by the challenge in an approach aimed at stimulating the development of histological image analysis methods for the accurate detection and classification of characteristics of tissue samples.

The confidence index was derived from the risk estimation vector generated by the last layer of the network. The softmax function is applied to the reversed risk (− risk vector), transforming the cost estimate into probabilities. This makes the confidence index easier to interpret and compare, without changing the order of the values. The confidence index was defined as the difference between the two highest probabilities of risk: if the probabilities were close, the network hesitated between two classes; if they were far apart, the network was considered more confident (see algorithm 1).

Algorithm 1: Grade-sensitive confidence
index (as defined by the applicant)

Input: Y ∈ [symbol] ⁿ, with n the number of classes
Output: u, the confidence index
Y ← softmax(−Y);
Y ← sort(Y; ascending = False);
U ← Y₀ − Y₁;

The confidence index was compared to the most popular methods in medical image analysis (“Monte Carlo Dropout Method” or “MC dropout”; and “Deep Ensembles”). As a reference, the raw output vector of the neural network is taken as the measure of confidence.

The “MC Dropout” model was trained with deactivation layers (i.e. “dropout layer”) with a rate r. During inference, M forward passes with deactivation layers were performed. Confidence was derived from the standard deviation of the Monte-Carlo samples generated according to the equation below:

$${U}_{unormalized} = \sqrt{\frac{{\sum }_{m = 0}^{M} ( {Y}_{m} - \overline{Y} )}{M - 1}}$$

With Y∈Rⁿ the output vector, n the number of classes, and M the number of MC samples. The standard deviation was inverted and normalised between 0 and 1 to be compared with the grade-sensitive confidence measurement (i.e. grade-sensitive confidence index).

For deep network sets, D networks were initialised with different seeds and trained independently of each other. The confidence index was calculated from the standard deviation of the D predictions made by the different networks in a similar way to the equation U_{unormalized}. Again, for comparison purposes, the standard deviation is inverted and normalised between 0 and 1.

The data set was divided into 5 training and validation sets in a stratified manner to perform a 5-fold cross validation. The folds were used to train and optimise the hyperparameters. The standard reference test was used for evaluation purposes only. The WSIs were cut into non-overlapping 224×224 pixel tiles at a resolution of 10× (1 μm/pixel) and the background tiles were removed, resulting in a total of 3.9 million tiles (from 2121 cuts). A DenseNet121 network was used to extract characteristics from the tiles (i.e. sub-images). This convolutional part of the network was frozen (i.e. the weights were defined as non-trainable) and initialised with pre-trained weights obtained from self-supervised learning. The self-supervised architecture was based on the SimCLR method. Dropout rates have been set at 0.5. Deep network sets or “Deep Ensembles” were obtained by training 5 models (D=5) with different seeds for the 5 data folds. 50 MC samples were generated (M=50) for the “MC Dropout” method. The predictions were made on the standard reference test set by averaging the output vectors of the D inferences (for D “Deep Ensembles”) or the M inferences (for “MC Dropout”), then averaging over the 5 folds. The models were trained for 100 epochs with an early stop on the validation sets. The RMSProp (Root Mean Square Propagation) optimizer was used with a momentum of 0.5 and a learning rate of 1e-4. In each training step, all tiles in the randomly sampled section were used. The implementation was carried out using TensorFlow (TF 2.8).

### Experiences and Results

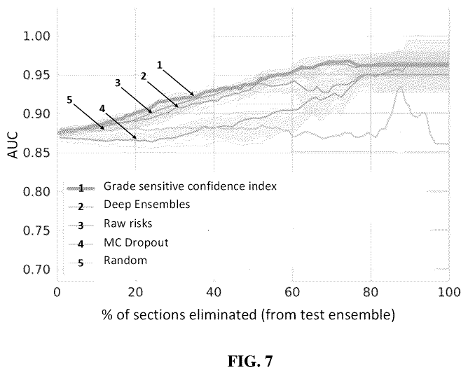

To compare the ability of the confidence measures to detect the most ambiguous cuts, classification performance is evaluated over the standard reference test set for a range of confidence threshold values. As the threshold increased, more cuts were considered uncertain by the model and removed from the test set. Metrics were then calculated on the remaining sections. The baseline was raw risk estimates obtained during training with the SOSR loss. It was compared to deep sets, “MC Dropout” and the grade-sensitive confidence index measure as defined by the applicant. The comparison was also made with the change of the AUC when the sections were chosen randomly (FIG. 7). It can be seen that the grade-sensitive measure, raw risk, and deep sets have similar behaviours: they consistently identify more difficult sections on which the model is more likely to make a poor prediction. On the other hand, “MC Dropout” fails to identify the most uncertain samples: the AUC remains constant when removing the first sections (the most uncertain according to the “MC Dropout” measure), similar to removing sections randomly.

In the case of screening use (e.g. using AI only if the prediction is reliable), a confidence threshold needs to be defined. In real cases, the threshold would be defined on the validation sets, depending on clinical constraints: reaching a negative predictive value (NPV). for the abnormal class (dysplasia+carcinoma)>0.95, for example. Here, to compare metrics on the same number of sections and ensure a fair comparison between sets and methods, the threshold is defined as the median of the confidence level. The distribution of confidence scores between classes revealed that confidence levels were always high for classes 0 and 3, which are in fact the easiest classes for pathologists to detect. On the other hand, confidence levels are often lower for intermediate classes (1 and 2), which are more difficult to classify, and the criteria for each class are difficult to assess. Table 6 shows that grade-sensitive measurement results in the largest differences between the subsets of low and high confidence sections.

TABLE 6
AUC for the standard reference test set stratified as a function of high and low confidence sections. Predictions
on the test set are obtained by averaging the predicted probabilities of the 5 training runs over the different
folds (“ensembling”), and a bootstrap (e.g. resampling by creating multiple data sets similar to the original
data set by randomly selecting samples with replacement) was performed to calculate the final metrics.

| | Threshold = median | | | | | | | | |
|---|---|---|---|---|---|---|---|---|---|
| | AUC | | | Carcinoma NPV | | | Abnormal NPV | | |
| | (average for 4 classes) | | | (3) | | | (1 + 2 + 3) | | |
| 1000 | Low level | High level | | Low level | High level | | Low level | High level | |
| bootstrap | of | of | | of | of | | of | of | |
| resampling | confidence | confidence | difference | confidence | confidence | difference | confidence | confidence | difference |
| MC | 0.842 | 0.884 | 4.2% | 0.950 | 0.936 | −1.4% | 0.664 | 0.733 | 6.90% |
| Dropout | | | | | | | | | |
| Raw risk | 0.797 | 0.934 | 13.70% | 0.920 | 1.0 | 8.0% | 0.621 | 0.742 | 12.10% |
| SOSR | | | | | | | | | |
| Deep | 0.790 | 0.928 | 13.8% | 0.938 | 1.0 | 6.2% | 0.540 | 0.809 | 26.90% |
| Ensembling | | | | | | | | | |
| Grade | 0.770 | 0.941 | 17.1% | 0.920 | 1.0 | 8.0% | 0.496 | 0.867 | 37.10% |
| sensitive | | | | | | | | | |
| confidence | | | | | | | | | |
| index | | | | | | | | | |

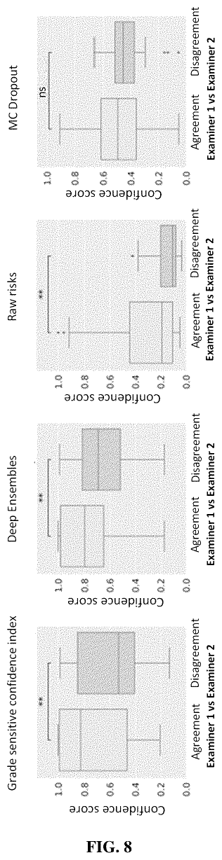

The applicant compared the confidence level with the results of the double-blind review of the standard reference test set. Confidence scores (i.e., confidence index) were measured for sections on which pathologists first agreed and sections on which pathologists disagreed during the blind review. Confidence scores for deep sets and grade-sensitive methods were significantly correlated with the review: higher confidence was associated with easier sections for pathologists (the ones they agreed on), and lower confidence with more difficult sections (the ones they disagreed on). FIG. 8 suggests that the confidence level of the model is correlated with the risk of disagreement between examiners (i.e. pathologists). It should be noted that the high variability of confidence scores is expected. Indeed, if the classification of a section was so difficult that the allocation was random, there would still be a 25% chance that the examiners would agree. If, in this case, the section received a low confidence score, this would result in a low confidence index for a section that the examiners agreed on. Since confidence scores measure different aspects of the prediction process (distance measurement, variance measurement, risk measurement), the applicant expects them to behave differently and vary over different ranges, but this does not negatively impact the quality of the score.

The grade-sensitive confidence index was compared with popular uncertainty measures for the difficult task of classifying precancerous lesions on histopathological samples. The grade-sensitive index, along with the deep set scores, accurately detected low-confidence samples, while the “MC Dropout” measurement did not perform well in the context of histological classification (FIG. 7). In addition, the grade-sensitive confidence index, as defined by the applicant, led to greater gains in classification performance between low and high confidence scores (Table 6), demonstrating that it is the most appropriate method for clinical practice among the techniques tested.

The grade-sensitive method is computationally-efficient and suitable for use in low-supervision workflows as it requires no annotation. This is unlike deep ensembles that require 4 additional models to be trained to assess confidence, thus multiplying training and prediction time. Given that the WSI classification is generally based on large data sets and requires long training times, this is a significant advantage of the method introduced by the applicant. Surprisingly, the raw risk estimates were a good assessment of confidence, stratifying the test ensemble between high and low confidence sub-ensembles almost as well as deep ensembles. This goes against the accepted theory that the softmax output of a network is not a good estimate of uncertainty. It is believed that this contradictory behaviour may be caused by the context of histological classification. Indeed, in other studies, uncertainty estimates were always calculated for binary tasks. In such a context, the softmax layer inevitably tends to force extreme decisions, with the subtleties necessary to stratify data based on their confidence being erased by this excessive behaviour. On the other hand, in the context of histological classification, the softmax layer may have more difficulty in forcing a class to a probability of 1; consequently, removing the softmax layer allows a better distribution of probabilities which can therefore be used more effectively to measure the confidence of a model.

It has been identified that the most uncertain sections according to the grade-sensitive confidence index measurement were mainly low grade (1) and high grade (2) lesions, i.e. intermediate classes. This observation corresponds to what pathologists experience in practice. Indeed, the histological classification system for precancerous lesions of the head and neck has always been controversial, and no fewer than 4 histological classification nomenclatures have been proposed in recent decades. This illustrates the inherent difficulty of the histological classification task, where boundaries between classes are subject to noise and pathologists' own calibration.

Finally, it was shown that measurements sensitive to grade, raw risks and deep ensembles were significantly correlated with agreements between pathologists (FIG. 8). This interesting finding demonstrates the portability of such measurements to clinical practice. Indeed, the confidence is able to detect sections on which pathologists will also struggle or even disagree. This can provide great assistance in detecting more difficult sections, either filtering them out or requesting a second opinion directly.

In conclusion, the present invention proposes a new approach for assessing the reliability of artificial intelligence models in the classification of WSI. This approach is easy to understand, cost-effective and takes into account the uncertainty inherent in histological samples. This contribution is thought to be an interesting step towards more reliable AI models for use in clinical practice, particularly for subjective tasks such as the classification of precancerous lesions.

### Example 7

### Cost Matrix

Classification errors do not have equally serious consequences. Consequently, a panel of pathologists established an assessment of each of these errors, i.e. they assigned each pair of possible results (i, j)∈{0.1.2.3}², a severity score of 0≤C_{i,j}≤1 (Table 7).

TABLE 7
Weighted accuracy error table - error table for weighting classification
errors based on their deviation from the ground truth

| | | | | Class 3: |
|---|---|---|---|---|
| | Class 0: | Class 1: | Class 2: | invasive |
| Ground | benign | low grade | high grade | carcinoma |
| truth | (prediction) | (prediction) | (prediction) | (prediction) |
| Class 0 | 0.0 | 0.1 | 0.7 | 1.0 |
| Class 1 | 0.1 | 0.0 | 0.3 | 0.7 |
| Class 2 | 0.7 | 0.3 | 0.0 | 0.3 |
| Class 3 | 1.0 | 0.7 | 0.3 | 0.0 |

### Example 8

In this example, the self-supervised model is based on SimCLR architecture. This architecture includes a characteristic extractor (DenseNet121) and a projection head (3 dense layers). The model was trained on 3.5 million 336×336 pixel tiles from the data set, with a batch size of 864, a temperature of 0.1, and a learning rate of 10e-4. The characteristic extractor was initialised with pre-trained weights on ImageNet and was trained for 5 epochs with the frozen characteristic extractor. Then, the model was trained for a total of 300 hours (143 epochs) using data augmentations such as cropping and resizing tiles to 224×224 pixels, random 90-degree rotations, flipping, customised hue augmentation, and adjustment of brightness, hue, contrast, and saturation.

### Example 9

The cost matrix used in the present invention reflects the medical impact that a misdiagnosis can have on patient care, recovery and follow-up. For example, according to experts, diagnosing a benign case (0) instead of a low-grade lesion (1) has less impact than diagnosing a high-grade lesion (2). In the absence of prior knowledge or experts to define error costs, more standard matrices with linear or quadratic weights, for example, could be used. The following results compare the performance achieved during training with the custom cost matrix and a linear cost matrix as defined below.

$$Linear cost matrix : [ \begin{matrix}0. & 0.33 & 0.66 & 1. \\ 0.33 & 0. & 0.33 & 0.66 \\ 0.66 & 0.33 & 0. & 0.33 \\ 1. & 0.66 & 0.33 & 0.\end{matrix} ]$$

TABLE 8
Classification performance by class (average +/− standard deviation
over the 5 folds) over the entire “gold standard test set”.

| | | | | Class 3: |
|---|---|---|---|---|
| | Class 0: | Class 1: | Class 2: | invasive |
| | benign | low grade | high grade | carcinoma |
| | (prediction) | (prediction) | (prediction) | (prediction) |
| AUC with a | 0.911 +/− | 0.78 +/− | 0.845 +/− | 0.963 +/− |
| custom matrix | 0.0078 | 0.0101 | 0.007 | 0.0071 |
| Total AUC = | | | | |
| 0.875 +/− | | | | |
| 0.005 | | | | |
| AUC with a | 0.91 +/− | 0.795 +/− | 0.849 +/− | 0.963 +/− |
| linear matrix | 0.0047 | 0.0171 | 0.0099 | 0.0068 |
| Total AUC = | | | | |
| 0.879 +/− | | | | |
| 0.0064 | | | | |

1-14. (canceled)

15. A computer-implemented method for assisting with the classification of a histological section from a patient into one of N classes with a predefined order, each class corresponding to a degree of severity of a pathology, N being an integer strictly greater than 2, by means of a confidence index representative of a degree of classification reliability, the method comprising:
implementing a machine learning model in order to classify said histological section in one of the N ordered classes, said model returning as output N classification probabilities each associated with one of the N ordered classes,
calculating the confidence index on the basis of the distance between the N classification probabilities,
wherein said machine learning model has been previously trained using a cost function weighted by a plurality of coefficients representative of a risk of incorrect classification, and wherein one of the plurality of coefficients has a higher value in a first case of incorrect classification in a class farther from a correct class, according to said predefined order, than in a second case of incorrect classification in a class less far from said correct class, according to said predefined order, than in the first case.

16. The computer-implemented method according to claim 15, characterised in that the distance between the two classification probabilities is a difference between the two highest classification probabilities of the N classification probabilities.

17. The computer-implemented method according to claim 15, wherein implementing the machine learning model comprises:
dividing said image into a set of M sub-images,
for each sub-image, extracting a plurality of characteristics,
for each sub-image, calculating a score based on the extracted plurality of characteristics,
classifying, based on the plurality of characteristics and the scores of the sub-images, said image into one of the N ordered classes.

18. The computer-implemented method according to claim 17, wherein the machine learning model is a multiple instance learning model.

19. The computer-implemented method according to claim 15, wherein the histological section comprises dysplastic lesions.

20. The computer-implemented method according to claim 19, wherein the dysplastic lesions are squamous lesions of the upper aerodigestive tracts.

21. The computer-implemented method according to claim 15, characterised in that the machine learning model is implemented using said cost function and in that each of the plurality of assignment probabilities is associated with a risk of incorrect classification, said risk varying in an increasing manner with said cost function.

22. The computer-implemented method according to claim 15, characterised in that calculating the score of each sub-image comprises implementing an attention mechanism.

23. The computer-implemented method according to claim 15, comprising, after calculating said confidence index, a comparison of said confidence index with a threshold value determined on the basis of a metric representative of reliability of the machine learning model calculated after implementing said model on a validation image database.

24. The computer-implemented method according to claim 23, wherein said metric is an area under the curve value.

25. The computer-implemented method according to claim 15, further comprising, after the comparison, an automatic assignment of a degree of analysis priority by a user, the degree of analysis priority being:
a degree of high priority if the confidence index is below the threshold value, in which case the analysis is assigned to an experienced user,
a degree of low priority if the confidence index is above the threshold value.

26. The computer-implemented method according to claim 15, further comprising an automatic assignment of a degree of analysis priority by a user, the degree of analysis priority being:
a degree of high priority if said histological section is classified into a class corresponding to a high degree of severity,
a degree of low priority if said histological section is classified into a class corresponding to a low degree of severity and has a high confidence index.

27. The computer-implemented method according to claim 15, characterised in that said histological section has been manually classified into one of the N ordered classes, denoted the manual class, by a user, prior to the implementation of the machine learning model, said method further comprising a comparison of the manual class with the class obtained during the implementation of the machine learning model.

28. A computer device comprising a circuit configured to implement a computer-implemented method according to claim 15.

29. A non-transitory computer-readable recording medium comprising instructions which, when executed by a computer, cause it to implement the computer-implemented method according to claim 15.
